(19) United States
(12) Patent Application Publication
(10) Pub. No.: US 2003/0176632 A1
(43) Pub. Date: Sep. 18, 2003

(54) Micromixing method for liquefied matter containing plural types of substance

(76) Inventor: Kazuo Nakamura, Saitama-City (JP)

Correspondence Address: Kazuo NAKAMURA, 8-7, Higashi-Takasagocho, Saitama Pref., Saitama-City, 336-0006, JP

(21) Appl. No.: 10/098,638

(22) Filed: Mar. 15, 2002

(51) Int. Cl. C08G 73/00; B01F 13/00; B01F 15/00; C07D 253/00; C07D 251/32

(52) U.S. Cl. 528/422; 366/348; 544/193

(57) ABSTRACT

To provide the micromixing method for liquefied matter containing plural types of substance which can be also applicable to promote chemical reaction among the plural types of substance with the micromixing when the plural types of substance are reactive each other.

Liquefied matter containing plural types of substance is promoted to mix in the molecule level by permeating through the numerous cylindrical micro-holes in the diameter from 0.03 to 100 micron meters formed in the phase separation porous body.

### BACK GROUND OF THE INVENTION

The invention is related with the technology of the highly precise mixing of liquefied matter containing plural types of substance to promote the mixing in the molecule level in order to shorten the mixing time, and of the micromixing of liquefied matter containing plural types of substance by causing chemical reaction among the plural types of substance which are chemically reactive with each other.

Evenly mixing process for liquefied matter containing plural types of substance has been widely carried out in many industries including chemical, food, mining, biochemical, medicine, and agricultural chemical industries, etc., but the mixing methods with mechanical mixers have been used in most of the processes. The mechanical mixing methods cannot necessarily provide effective and sufficient mixing result, and furthermore accompanies vibrations and noises from the driving mechanism of the mixers. The worst part of the mixing result with the mechanical mixing is the mixture of the large groups of molecule masses.

The static mixer without the driving mechanism with the use of special shape mixing blades such as the helical blade is available, but this cannot provide precise and effective mixing result either and can mix plural types of substance in the level of large mass of molecules at best, and precise micromixing is not possible with these mixers.

On the other hand, if liquefied matter containing plural types of substance is precisely mixed to result in the mixture of the plural types of substance in the molecule level, it is expected that the molecule level mixing causes enough collisions between molecules to cause chemical reaction among the plural types of substance. The molecule level mixing is further expected to comparatively easily cause chemical reactions that were possible under the high temperature, high pressure, long reaction time or using particular catalyst in the past.

The purpose of the invention is to eliminate vibrations and driving mechanism inherent in the existing mixers, and to achieve the very precise molecule level mixing not possible with the existing mixing methods, resulting in the shorter mixing time. Furthermore the invention is to provide the molecule level precise and micromixing of liquefied matter containing chemically reactive plural types of substance resulting and promoting chemical reaction among the plural types of substance.

### SUMMARY OF THE INVENTION

The invention is a result of studies to achieve the above purposes. I discovered in the studies that chemical reaction was greatly promoted among the plural types of substance contained in liquefied matter when such substances are chemically reactive each other as the solution was precisely mixed in the molecule level during permeation through microholes in the diameter of 0.03 to 100 micron meters formed in the phase separation porous body such as phase separation porous glass body.

The inventor of this invention was the first to discover the phenomenon described above. The phenomenon can be utilized to eliminate vibration and noises cause by the driving mechanism for more precise and more effective mixing, and to efficiently advance some types of chemical reactions such as those possible only under high temperature, high pressure, long reaction time, or using particular catalyst.

Although I have not clarified the mechanism to result in the effective mixing as described above by permeating liquefied matter containing plural types of substance through microholes in the phase separation porous body having the porous characteristics as described above, the microholes in the phase separation porous body have cylindrical shape with very narrow distribution of pore size, and in some cases, branch off and join together inside the porous body as a result of the particular phase separation porous body producing method. The plural types of substance contained in liquefied matter collide with each other in the molecule level and are mixed during permeation through the cylindrical microholes. If the phase separation porous glass body is used particularly, the plural types of substance repeatedly collide in the complicated patterns with the hard microhole walls made of the phase rich in SiO2 while permeating through the cylindrical microholes to achieve excellent mixing result.

Permeation of liquefied matter containing plural types of substance through the phase separation porous body of this invention can be compared with permeation of the solution through sintered porous glass body or sintered porous aluminum body as the sintered porous body has ink bottle shape microholes having the broad distribution of pore size in comparison with the cylindrical shape for the phase separation porous body. Consequently, precise and effective mixing result possible with the phase separation porous body of this invention cannot be obtained with the sintered porous body for the same liquefied matter containing plural types of substance. Therefore, the purpose of this invention can only be achieved when liquefied matter containing plural types of substance is permeated through the afore-mentioned cylindrical microholes in the phase separation porous body.

Therefore, the summary of this invention is as follows:

(1) Micromixing method for liquefied matter containing plural types of substance by permeating the liquefied matter through numerous microholes in the diameter of 0.03 to 100 micron meter in the phase separation porous body to promote mixing of plural types of substance in the molecule level.

(2) Micromixing method for liquefied matter containing plural types of substance using the afore-mentioned phase separation porous body made of the phase separation porous glass material in the hollow cylindrical shape

(3) Micromixing method for the plural types of substance contained in liquefied matter referred to the above (1) or (2) by permeating the liquefied matter through microholes in the afore-mentioned phase separation porous glass body in the hollow cylindrical shape using the differential pressure

(4) Micromixing method for liquefied matter containing plural types of substance referred to in the above (1), (2), or (3) using the afore-mentioned phase separation porous glass body having the cylindrical shape microholes formed by separating molded borosilicate glass into a phase rich in SiO2 and another phase rich in B2O3 and CaO in the heat treatment, and then eluting the phase rich in B2O3 and CaO in the acid treatment

(5) Micromixing method for liquefied matter containing plural types of substance referred to in the above (1), (2), (3), or (4) by causing chemical reaction among the plural types of substance contained in the liquefied matter with the micromixing.

(6) Micromixing method for the afore-mentioned liquefied matter containing one or plural types of monomers referred to in the above (1), (2), (3), (4), or (5), which are polymerized to produce oligomer or polymer

(7) Micromixing method for solution containing plural types of substance referred to in the claim 6 that monomer solution is ethyl benzimidate solution in tetrahydrofuran (THF) and that the oligomer is 2,4,6 triphenyl 1,3,5 triazine which is trimer of benzonitrile.

### BRIEF DESCRIPTION OF THE DRAWING

FIG. 1 is an enlarged view of the cross section of the cylindrical microholes in the phase separation porous glass body in the forming process with (a) showing the phase separating aspect, and (b) the aspect forming microholes with the phase rich in B2O3 and CaO eluted.

FIG. 2 is an outside view of the phase separation porous body formed in the hollow cylindrical type.

### DESCRIPTION OF THE NUMBERED PARTS

12 Phase separation porous glass body

13 Borosilicate glass

14 Phase rich in SiO2

15 Phase rich in B2O3 and CaO

16 Cylindrical through hole

17 Wall having the microholes

21 Hollow cylindrical material

22 Periphery surface

23 Hollow part

### DETAILED DESCRIPTION OF THE INVENTION

Liquefied matter containing plural types of substance for the micromixing of this invention is made of more than two different types of substance. Solvent in solution or in dispersion can be considered as one type of substance. Besides matter in the liquid form in the room temperature, matter which can be changed into the liquid form when heated can also be considered as a liquefied matter permeated in this invention. One type of substance dissolved or dispersed in one type of solvent, more than two types of substance dissolved or dispersed in one type of solvent, and one type of substance dissolved or dispersed in two types of solvent are some examples of liquefied matter containing plural types of substance referred to in this invention. It is found desirable that the diameter of substance dispersed in liquefied matter is below half of the diameter of microholes in the phase separation porous body, because the substance must permeate through microholes in the phase separation porous body.

The application examples of the method of this invention are to produce very uniformly mixed liquefied matter or dispersion by dissolving or dispersing one or more than two types of substance in liquid, to produce uniformly mixed liquefied matter by mixing more than two types of substance in liquid, to mix more than two types of chemical substance in liquid to cause chemical reaction, to produce uniform mixture of aqueous liquid and oily liquid, and to cause chemical reaction with phase-transfer catalyst. The more concrete application examples of this invention are promotion of chemical reaction, promotion of solution polymerization, and production of salad dressing made of oil and water, of paint made of plural number of components, of liquid fertilizer or agricultural chemicals, and of polymer or oligomer which are in liquid form during reaction.

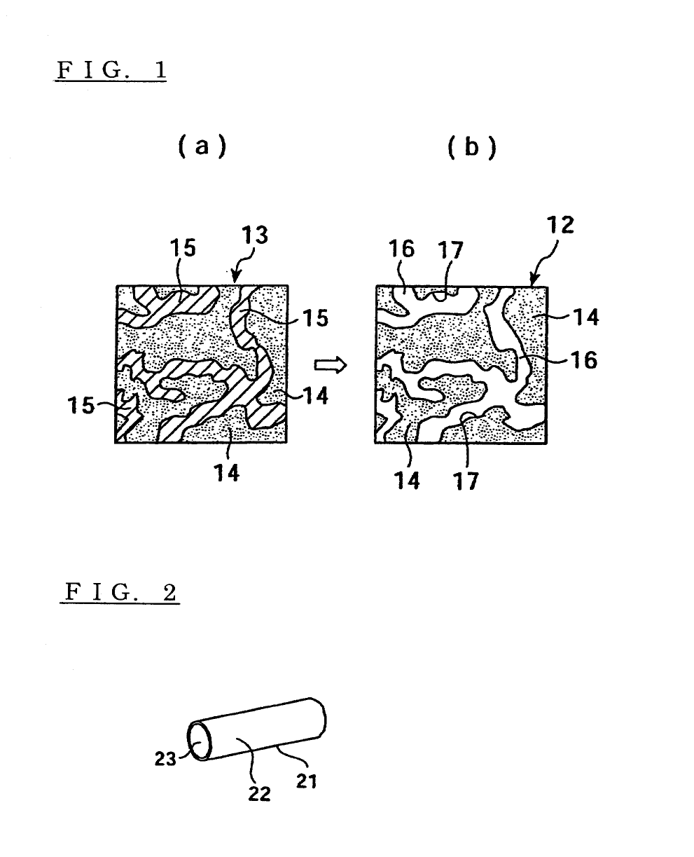

The phase separation porous body having particular microhole characteristics of this invention can be made of inorganic material such as glass, silica, and metal, etc. or organic material such as polymer, etc. to permeate liquefied matter containing afore-mentioned substance of more than one type through the microholes in the phase separation porous body. FIGS. 1(a) and 1(b) show the desirable example of the enlarged cross section of the microholes in the phase separation porous glass body 12 in the forming process to be used for the invention. Borosilicate glass 13 is separated into the phase 14 rich in SiO2 and phase 15 rich in B2O3 and CaO in the heat treatment as shown in FIG. 1(a), and then the acid treatment is given to elute the phase 15 rich in B2O3 and CaO. Numerous microholes are formed with walls 17 made of the phase 14 rich in SiO2 after the phase 15 rich in B2O3 and CaO is eluted as shown in FIG. 1(b).

The afore-mentioned phase separation porous glass having numerous microholes referred to in this invention has been already known. For instance, it was mentioned in the detailed description for U.S. Pat. No. 4,657,875. It is also known that the microholes in the phase separation porous glass can be formed in various diameters in the range from 4 nm to 20 micron meters depending on the glass composition and the phase separation condition such as the time and temperature.

The diameter (means the average diameter for this invention) of the microholes in the phase separation porous body typically represented by the phase separation porous glass body used in this invention plays the important role to determine how well the mixing result can be achieved, which is the main purpose of this invention. I found the diameter in the range of 0.03 to 100 micron meters for the microholes in the phase separation porous body was required to effectively fulfill the purpose of this invention. The microholes smaller than 0.03 micron meters in diameter provide too big resistance for solution to permeate through the microholes, while the microholes in the diameter larger than 100 micron meters reduce the molecule level mixing effect, resulting in the purpose of this invention not fulfilled. I found the desirable microhole diameter was in the range from 0.3 to 20 micron meters, and the best diameter range was from 1.0 to 10 micron meters.

The afore-mentioned phase separation porous body can be in various shapes such as a board shape, hollow cylindrical shape, cylindrical shape, or grain shape, etc. to be used for the purpose of this invention, but the hollow cylindrical shape is desirable if the mixing efficiency is taken into consideration. FIG. 2 shows the outside view of the phase separation porous body 21 formed in the hollow cylindrical shape with the outside diameter of 3 to 15 mm, and wall thickness of 0.2 to 1.5 mm. The microholes in the phase separation porous body 21 has microholes formed to go through the wall thickness from the periphery 22 toward the inside hollow space 23 or from the inside hollow space 23 toward the periphery 22.

In applying the method of this invention using the phase separation porous body, liquefied matter containing plural types of substance is permeated through microholes in the wall of the hollow cylindrical shape of the phase separation porous body from the inside hollow space toward the periphery, or vice versa. It is desirable to provide a differential pressure between the inlet and outlet of the microholes so for liquefied matter containing plural types of substance as to efficiently permeate through the microholes.

To provide the differential pressure, the gravity can be applied to the liquefied matter, a pressure applied on the inlet side, or negative pressure on the outlet side by sucking the liquefied matter in. Considering the physical strength of the hollow cylindrical body, it is advisable to feed liquefied matter containing plural types of substance from the periphery of the hollow cylindrical body toward the hollow inside.

The phase separation porous body and/or liquefied matter can be heated and kept hot as necessary to promote mixing of the liquefied matter or to prevent the liquefied matter from solidifying when the liquefied matter containing plural types of substance is permeated through the phase separation porous body. The temperature to heat the liquefied matter varies depending on the type of liquefied matter or the purpose of mixing, but is usually in the range from 25 degrees C. to 120 degrees C. Permeation of liquefied matter containing plural types of substance through the phase separation porous body should not necessarily be once, but can be more than twice as necessary depending on the necessary mixing level.

### APPLICATION EXAMPLE

One application example of this invention for mixing a particular liquefied matter containing plural types of substance is introduced as follows, but the invention should not be understood to be applicable only to applications similar to the example.

2,4,6-triphenyl-1,3,5-triazine was synthesized by trimerization of ethyl benzimidate C6H5C (═NH) OCH2CH3, 1.5 g (10 mmol) in tetrahydrofuran (THF) solution. The solution prepared from ethyl benzimidate (1.5 g, 10 mmol), glacial acetic acid (0.66 g, 11 mmol) and THF (5 ml) was passed through microholes of the phase separation porous body for one minute at room temperature. The solution passed through was then analyzed with a High Pressure Liquid Chromatography (HPLC) to determine the product.

The phase separation porous body used for the mixing test was the phase separation porous glass body in the hollow cylindrical shape (outside diameter of 10 mm, wall thickness of 0.8 mm, and length of 115 mm) having 2.35 micron meter diameter cylindrical microholes with one open end of the porous body closed, and the afore-mentioned mixed liquid introduced from the other open end for the liquid to permeate through the microholes in the wall of the hollow cylindrical body and to collect the permeated liquid from the periphery.

The above process was repeated for 240 times (took 4 hours in total for the permeation). The production of 2,4,6-triphenyl-1,3,5-triazine was confirmed after one permeation, and the yield to change to trimer reached 42% after 120 times of permeation, and 55% after 240 times of the permeation. The collected liquid was distilled to eliminate the solvent tetrahydrofuran, and then washed with methanol to obtain 0.57 g of the trimer 2,4,6-triphenyl-1,3,5-triazine of 235 to 237 degrees C. melting point. The resultant trimerization yield was 55%.

As a result of the above test, it is confirmed that the permeation process through the hollow cylindrical phase separation porous glass body can provide molecule level mixing to greatly promote chemical reaction to change raw material of ethyl benzimidate to trimer of benzonitrile. THF solution containing ethyl benzimidate and glacial acetic acid, prepared in the same manner in the example B, was vigorously stirred for 4 hours at room temperature in a flask using a magnetic stirrer. The reaction solution was then analyzed with HPLC, confirming that the trimeric 2,4,6-triphenyl-1,3,5-triazine was formed from ethyl benzimidate.

The resultant liquid was distilled to eliminate the solvent THF, and then washed with methanol to obtain 0.08 g of 2,4,6-triphenyl-1,3,5-triazine. The trimerization yield was only 8%.

The comparison between the above two test results shows that the yield of the obtained 2,4,6-triphenyl-1,3,5-triazine in the latter test was very low of about 1/7 of the yield from the former test during the same reaction time.

As described above, this invention is different from the existing mixing method, and provides the micromixing method for liquefied matter containing plural types of substance enabling very precise mixing results in the molecule level without any driving mechanism accompanying vibration and noises.

This invention also provides the micromixing method for liquefied matter containing plural types of substance which cause chemical reaction among the plural types of substance contained in the liquefied matter because of the molecule level mixing as described above.

What is claimed is:

1. Micromixing method to mix plural types of substance contained in liquefied matter by permeating the liquefied matter through a phase separation porous body having numerous microholes in the diameter of 0.03 to 100 micron meters to promote the mixing in the molecule level.

2. Micromixing method for plural types of substance contained in liquefied matter with the use of the phase separation porous glass body in the hollow cylindrical shape referred to in the above claim 1.

3. Micromixing method for plural types of substance contained in liquefied matter referred to in the above claims 1 and 2 by permeating the liquefied matter through the numerous microholes in the hollow cylindrical phase separation porous glass body with the differential pressure.

4. Micromixing method for liquefied matter containing plural types of substance referred to the above claim 1, 2, or 3 with the use of the above mentioned phase separation porous glass body mainly made of SiO2, which is formed by separating molded borosilicate glass into a phase rich in SiO2 and another phase rich in B2O3 and CaO, and processing them in acid to have the phase rich in B2O3 and CaO to elute, and the obtained microholes have cylindrical hole structure.

5. Micromixing method for liquefied matter containing plural types of substance referred to in the above claim 1, 2, 3, or 4 to cause chemical reaction among the plural types of substance.

6. Micromixing method to produce oligomers or polymers with the micromixing of liquefied matter containing one or more types of monomers referred to in the above claim 1, 2, 3, or 4.

7. Micromixing method for solution containing plural types of substance referred to in the claim 6 that monomer solution is ethyl benzimidate solution in tetrahydrofuran(THF) and that the oligomer is 2,4,6 triphenyl-1,3,5 triazine which is trimer of benzonitrile.
